(19) United States
(12) Patent Application Publication
(10) Pub. No.: US 2020/0399250 A1
(43) Pub. Date: Dec. 24, 2020

(54) Pyrimidine Derivatives as Tropomyosin Receptor Kinase A (TRKA) Inhibitors

(71) Applicant: BIOMED X GMBH, Heidelberg (DE)

(72) Inventors: Eid Sameh, Heidelberg (DE); Simone Fulle, Heidelberg (DE); Benjamin Merget, Schriesheim (DE); Samo Turk, Hamburg (DE)

(21) Appl. No.: 16/764,996

(22) PCT Filed: Nov. 22, 2018
(86) PCT No.: PCT/EP2018/082183

§ 371 Date: May 18, 2020

(30) Foreign Application Priority Data
Nov. 23, 2017 (EP) 17203290.6

(51) Int. Cl. C07D 403/14; A61P 25/00; A61P 35/00

(52) U.S. Cl. CPC C07D 403/14; A61P 35/00; A61P 25/00

(57) ABSTRACT

The present invention relates to novel TrkA inhibitors of formula (1) which are useful in the treatment or prevention of acute and chronic pain but also for other abnormal activities of TrkA beyond pain therapy, such as inflammation and cancer.

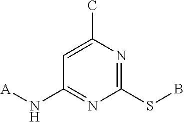

The present invention relates to novel compounds of Formula (1) that are TrkA inhibitors and are useful in the treatment or prevention of acute and chronic pain but also for other abnormal activities of TrkA beyond pain therapy, such as inflammation and cancer.

### BACKGROUND OF THE INVENTION

Based on the physical causes, pain can be divided into three types: nociceptive, neuropathic, and mix-type.

Nociceptive pain is the term for pain that is detected by nociceptors. Nociceptors are free nerve endings that terminate just below the skin, in tendons, in joints, and in internal organs. Nociceptive pain typically responds well to treatment with opioids and NSAIDs. There are several types of nociceptive pain: somatic pain, visceral pain, and cutaneous pain. Visceral pain comes from the internal organs. Deep somatic pain is initiated by stimulation of nociceptors in ligaments, tendons, bones, blood vessels, fascies and muscles, and is dull, aching, poorly localized pain. Examples include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns. Nociceptive pain is usually short in duration and ends when the damage recovers. Examples of nociceptive pain include postoperative pain, sprains, bone fractures, burns, bumps, bruises, and inflammatory nociceptive pain. Inflammatory nociceptive pain is associated with tissue damage and the resulting inflammatory process.

Neuropathic pain is produced by damage to the neurons in the peripheral and central nervous systems and involves sensitization of these systems. Because the underlying etiologies are usually irreversible, most of neuropathic pain are chronic pain. Most people describe neuropathic pain as shooting, burning, tingling, lancinating, electric shock qualities, numbness, and persistent allodynia. The nomenclature of neuropathic pain is based on the site of initiating nervous system with the etiology; for examples, central post-stroke pain, diabetes peripheral neuropathy, post-herpetic (or post-shingles) neuralgia, terminal cancer pain, phantom limb pain.

Mix-type pain is featured by the coexistence of both nociceptive and neuropathic pain. For example, muscle pain trigger central or peripheral neuron sensitization leading to chronic low back pain, migraine, and myofascial pain.

Receptor tyrosine kinases (RTKs) are a sub-family of protein kinases that play a critical role in cell signaling and also are involved in a variety of processes related to nerve activity. These include pain transmission in the spinal cord as well as in the peripheral nerve endings where the pain signal starts.

The tyrosine kinase receptor (Trk) family members are high affinity receptors of the RTK class and are activated by a group of soluble growth factors called neurotrophins (NT). The Trk receptor family has three members-TrkA, TrkB and TrkC. Among the neurotrophins are (i) nerve growth factor (NGF) which activates TrkA, (ii) brain-derived neurotrophic factor (BNDF) and NT-4/5 which activates TrkB, and (iii) NT3 which activates TrkC. Trks are widely expressed in neuronal tissue and are implicated in the maintenance, signaling and survival of neuronal cells (Patapoutian, A. et al, Current Opinion In Neurobiology, 2001, 11, 272-280). Recent literature also indicates that activation of TrkA with NGF causes downstream upregulation of certain ion channels which are important in increasing the electric signaling from the nerve endings which experience the inflammation, thus inducing pain (e.g. VR-1, Winston et al. Pain 2001,89,181; sodium channels, Choi et al. Molecular and Cellular Biology 2001, 21, 2695; ASIC, Mamet et al. Journal of Biological Chemistry 2003, 278, 48907). Binding of Nerve Growth Factor (NGF) to TrkA triggers TrkA-dimerization and activation of downstream signaling pathways linked to neuronal development and nociception (Khan, N. et al., Molecules 20, 10657-88 (2015)).

Each Trk receptor contains an extra-cellular domain (ligand binding), trans-membrane region and intra-cellular domain (including kinase domain). Upon binding of the ligand, the kinase domain catalyzes the auto-phosphorylation and triggers downstream signal transduction pathways.

Over the last 10 years many scientific reports have been published which link Trk signaling with induction of pain. Inhibitors of the Trk/neurotrophin pathway have been demonstrated to be effective in numerous pre-clinical animal models of pain. For example, antagonistic NGF and TrkA antibodies (e.g., RN-624) have been shown to be efficacious in inflammatory and neuropathic pain animal models and in human clinical trials (Woolf, C. J. et al. (1994) Neuroscience 62, 327-331; Zahn, P. K. et al. (2004) J. Pain 5, 157-163; McMahon, S. B. et al., (1995) Nat. Med. 1, 774-780; Ma, Q. P. and Woolf, C. J. (1997) Neuroreport 8, 807-810; Shelton, D. L. et al. (2005) Pain 116, 8-16; Delafoy, L. et al. (2003) Pain 105, 489-497; Lamb, K. et al. (2003) Neurogastroenterol. Motil. 15, 355-361; Jaggar, S. I. et al. (1999) Br. J. Anaesth. 83, 442-448). Additionally, literature indicates that after inflammation, BDNF levels and TrkB signaling is increased in the dorsal root ganglion (Cho, L. et al. Brain Research 1997, 749, 358) and several studies have shown antibodies that decrease signaling through the BDNF/TrkB pathway inhibit neuronal hypersensitization and the associated pain (Chang-Qi, L et al. Molecular Pain 2008, 4:27). A very recent article reports that TrkA is a validated drug target for cancer and pain (Norman, B. H. et al. J. Med. Chem. 60, 66-88 (2017)). In addition, pain suppression effect of TrkA inhibitors has been shown in in-vitro models (Nwosu, L. N., et al., Ann. Rheum. Dis. annrheumdis-2014-207203 (2015). doi:10.1136/annrheumdis-2014-207203; Andrews, S. W., Array biopharma Available at: http://www.arraybiopharma.com/files/6313/9810/8021/PubAttachment587.pdf). A recent publication shows that loss-of-function TrkA variants are associated with congenital insensitivity to pain (Bakri, F. G. et al. Clin. Case Reports 4, 997-1000 (2016)).

There are few reports of selective Trk tyrosine kinase inhibitors that are highly selective for TrkA and TrkB. Cephalon describes CEP-751, CEP-701 (George, D. et al Cancer Research, 1999, 59, 2395-2401) and other indolocarbazole analogs (WO0114380) as Trk inhibitors. It was shown that the alkaloid K252a, which is related to CEP-701/751, when injected into rats with pancreatitis could suppress mechanical hypersensitivity (Winston et al. Journal of Pain 2003, 4, 329).

In addition, it has been shown that tumor cell invading macrophages directly stimulate TrkA located on peripheral pain fibers. Using various tumor models in both mice and rats it was demonstrated that neutralizing NGF with a monoclonal antibody inhibits cancer related pain to a degree similar or superior to the highest tolerated dose of morphine. In addition, activation of the BDNF/TrkB pathway has been implicated in numerous studies as a modulator of various types of pain including inflammatory pain (Matayoshi, S., J. Physiol. 2005, 569:685-95), neuropathic pain (Thomson, S. W., Proc. Natl. Acad. Sci. USA 1999, 96:7714-18) and surgical pain (Li, C.-Q. et al., Molecular Pain, 2008, 4(28), 1-11). Because TrkA and TrkB kinases may serve as a mediator of NGF driven biological responses, inhibitors of TrkA and/or other Trk kinases may provide an effective treatment for chronic pain states.

Recent literature has also shown that overexpression, activation, amplification and/or mutation of Trks are associated with many cancers including neuroblastoma (Brodeur, G. M., Nat. Rev. Cancer 2003, 3, 203-2016), ovarian cancer (Davidson. B., et al., Clin. Cancer Res. 20039, 2248-2259), breast cancer (Kruettgen et al, Brain Pathology 2006, 16: 304-310), prostate cancer (Dionne et al, Clin. Cancer Res. 1998,4(8): 1887-1898), pancreatic cancer (Dang et al, Journal of Gastroenterology and Hepatology 2006, 21(5): 850-858), multiple myeloma (Hu et al, Cancer Genetics and Cytogenetics 2007, 178: 1-10), astrocytoma and medulloblastoma (Kruettgen et al, Brain Pathology 2006, 16: 304-310), glioma (Hansen et al, Journal of Neurochemistry 2007, 28(3)221-229), lung adenocarcinoma (Perez-Pinera et al, Molecular and Cellular Biochemistry 2007, 295(1&2), 19-26), large cell neuroendocrine tumors (Marchetti et al, Human Mutation 2008, 29(5), 609-616), and colorectal cancer (Bardelli, A., Science 2003, 300, 949). In preclinical models of cancer, Trk inhibitors are efficacious in both inhibiting tumor growth and stopping tumor metastasis. In particular, non-selective small molecule inhibitors of Trk A, B and C and Trk/Fc chimeras were efficacious in both inhibiting tumor growth and stopping tumor metastasis (Nakagawara, A. (2001) Cancer Letters 169:107-114; Meyer, J. et al. (2007) Leukemia, 1-10; Pierottia, M. A. and Greco A., (2006) Cancer Letters 232:90-98; Eric Adriaenssens, E. et al. Cancer Res. (2008) 68(2) 346-351) (Truzzi et al, Journal of Investigative Dermatology 2008, 128(8): 2031-2040. Therefore, an inhibitor of the Trk family of kinases is expected to have utility in the treatment of cancer.

In addition, inhibition of the neurotrophin/Trk pathway has been shown to be effective in treatment of pre-clinical models of inflammatory diseases. For example, inhibition of the neurotrophin/Trk pathway has been implicated in pre-clinical models of inflammatory lung diseases including asthma (Freund-Michel, V; Frossard, N.; Pharmacology & Therapeutics (2008), 117(1), 52-76), interstitial cystitis (Hu Vivian Y; et. al. The Journal of Urology (2005), 173(3), 1016-21), inflammatory bowel diseases including ulcerative colitis and Crohn's disease (Di Mola, F. F, et. al., Gut (2000), 46(5), 670-678) and inflammatory skin diseases such as atopic dermatitis (Dou, Y.-C.; et. al. Archives of Dermatological Research (2006), 298(1), 31-37), eczema and psoriasis (Raychaudhuri, S. P.; et. al. Journal of Investigative Dermatology (2004), 122(3), 812-819).

Inflammation is a process by which microbes or tissue injury induce the release of cytokines and chemokines from various cell types producing increased blood vessel permeability, upregulation of endothelal receptors, and thus increased egress of various cells of the innate and adaptive immune system which enter surrounding tissue and grossly produce the classical picture of inflammation, i.e. redness, swelling, heat and pain.

Inflammation is a localized reaction of live tissue due to an injury, which may be caused by various endogenous and exogenous factors. The exogenous factors include physical, chemical, and biological factors. The endogenous factors include inflammatory mediators, antigens, and antibodies. Endogenous factors often develop under the influence of an exogenous damage. An inflammatory reaction is often followed by an altered structure and penetrability of the cellular membrane. Endogenous factors, such as mediators and antigens define the nature and type of an inflammatory reaction, especially its course in the zone of injury. In the case where tissue damage is limited to the creation of mediators, an acute form of inflammation develops. If immunologic reactions are also involved in the process, through the interaction of antigens, antibodies, and auto-antigens, a long-term inflammatory process will develop. Various exogenous agents, for example, infection, injury, and radiation, also provide the course of inflammatory process on a molecular level by damaging cellular membranes which initiate biochemical reactions.

The current treatment regimens for pain conditions utilize compounds which exploit a very limited range of pharmacological mechanisms. One class of compounds, the opioids, stimulates the endogenous endorphin system. Morphine is an example of this class. Compounds of the opioid class have several drawbacks that limit their use, e.g. emetic and constipation effects, and negative influence on respiratory capability. Their use is also restricted because of their addiction liabilities. The second major class of analgesics, the non-steroidal anti-inflammatory analgesics of the COX-1 or COX-2 types, also have liabilities such as insufficient efficacy in severe pain and inflammatory conditions and at long term use the COX-1 inhibitors cause ulcers of the mucosa.

Thus, there is a need for new active agents useful for the treatment of pain and inflammatory conditions.

### DESCRIPTION OF THE PRESENT INVENTION

The present invention relates to compounds of Formula (1)

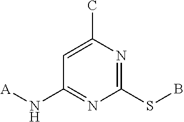

wherein
A: 5-membered monocyclic aromatic heterocyclic ring or 8-10 membered bicyclic aromatic heterocyclic ring each containing 2-4 nitrogen atoms and optionally one sulfur or oxygen atom, said ring optionally substituted (a) with one or more C₁-C₄ alkyl or alkenyl groups, or (b) with a 5- or 6-membered alkyl ring or alkenyl ring or aryl ring that are optionally substituted with one or more halogen or C₁-C₄ alkyl or alkenyl groups including mono- or poly-halogenated C₁-C₄ alkyl or alkenyl groups
B: methylene-aryl or aryl rings that are optionally substituted with one or more halogen or C₁-C₄ alkyl or alkenyl groups including mono- or poly-halogenated C₁-C₄ alkyl or alkenyl groups
C: Monocyclic or bicyclic, saturated or monounsaturated or polyunsaturated or aromatic heterocycles having 5-10 ring atoms among them 1-5 heteroatoms which are preferably N, O and S, substituted, where appropriate, once, twice or thrice with residues R^{§}
R^{§}: —OH, —SH, —C₁-C₄ alkyl, —O—C₁₋₈ alkyl, —O—C₆₋₁₄ aryl, —S—C₁₋₄ alkyl, —S—C₆₋₁₄ aryl, —SO—C₁₋₄ alkyl, —SO—C₆₋₁₄ aryl, —SO₂—C₁₋₄ alkyl, —SO₂—C₆₋₁₄ aryl, —SO₃H, —OSO₂C₁₋₈ alkyl, —OSO₂C₆₋₁₄ aryl, —COOH, —COOC₁₋₈ alkyl, —(CO)C₁₋₈ alkyl, —COOH, —CONH₂, —CONHC₁₋₆ alkyl, —CON(C₁₋₆ alkyl)₂. —NH₂, —NHC₁₋₆ alkyl, —N(C₁₋₆ alkyl)₂, —NHC₆₋₁₄ aryl, —NH-hetaryl, —N(C₆₋₁₄ aryl)₂, —N(C₁₋₆ alkyl)(C₆₋₁₄ aryl), —C₁₋₆ alkyl, —C₂₋₁₂ alkenyl, halogen, —CH₂CH₂OH, —CH₂CH₂SH, —CH₂CH₂SCH₃, -sulfamoyl, alkylsulfamoyl, dialkyl sulfamoyl, -sulfo, phosphono, —CN, —NO₂ and/or —SCN
as well as pharmaceutically compatible salts, solvates, active metabolites, tautomers and prodrugs of these compounds.

As used herein, the following definitions shall apply unless otherwise indicated.

The phrase “optionally substituted” is used interchangeably with the phrase “substituted or unsubstituted” or with the term “(un)substituted”. Unless otherwise indicated, an optionally substituted group may be a substituent at each substitutable position of the group, and each substitution is independent of the other.

The term “C₁-C₄ alkyl or alkenyl” designates both the unbranched and branched, as well as saturated and unsaturated functional aliphatic groups. In particular, it means methyl, ethyl, n-propyl, isopropyl, n-butyl, sec-butyl, isobutyl, tert-butyl, ethenyl (vinyl), propenyl, or butenyl.

“5- or 6-membered alkyl or alkenyl ring” means cyclopentyl, cyclohexyl, cyclopentenyl or cyclohexenyl. The alkyl- or alkenyl ring can be substituted, where appropriate, with one or several halogen substituents or C₁-C₄ alkyl or alkenyl groups including mono- or poly-halogenated derivatives thereof.

“Aryl” refers to either an aromatic 5-6 membered monocyclic carbocyclic or heterocyclic ring system, or a 8-10 membered bicyclic carbocyclic or heterocyclic ring system, including heteroaromatic systems with 1-5 hetero atoms (N, O or S), preferably 1, 2 or 3 heteroatoms.

The aryl ring can be substituted, where appropriate, with one or several halogen substituents or C₁-C₄ alkyl or alkenyl groups including mono- or poly-halogenated derivatives thereof. The preferred aryl group is a carbocyclic ring, e.g. phenyl, benzyl, tolyl, xylyl or naphthyl. Examples for preferred “heteroaromatic rings” or “heteroaryls” are pyrazinyl, pyridinyl, furanyl, thienyl, thiazinyl, thiophenyl, pyrimidinyl, isoxazolyl, isothiazolyl, oxazolyl, thiazolyl, pyrazolyl, pyrrolyl, oxadiazolyl, pyridyl, benzoimidazolyl, benzothienyl, imidazolyl, triazolyl, tetrazolyl or benzothiazolyl.

A “5-membered monocyclic aromatic heterocyclic ring or 8-10 membered bicyclic aromatic heterocyclic ring each containing 2-4, preferably 2 or 3, nitrogen atoms and optionally one sulfur or oxygen atom” comprises, for example, a imidazolyl, pyrazolyl, triazolyl, pyrazolo[1,5-]pyridinyl, pyrazolopyrimidinyl, benzoimidazolyl, imidazopyridinyl, purine or indolyl ring.

“Halogen” means fluorine, chlorine, bromine or iodine.

“Mono- or poly-halogenated alkyl or alkenyl groups” means an alkyl or alkenyl group substituted with one or more halogen atoms, e.g. CH₂F, CHF₂ or CF₃.

“Monocyclic or bicyclic, saturated or monounsaturated or polyunsaturated or aromatic heterocycles having 5-14 ring atoms among them 1-5 heteroatoms which are preferably N, O and S, substituted, where appropriate, once, twice or thrice with residues R^{§}” preferably comprise the following groups: methylpiperazinyl, thienyl, pyridinyl, pyrimidinyl, piperazinyl, pyridyl, isoxazolyl, piperidinyl, pyrazinyl, morpholino, pyrrolyl, triazinyl, tetrazolyl, oxazolyl, benzo[d][1,3]dioxolyl, indolyl, imidazolyl, pyrazolyl, furanyl. Monocyclic or polycyclic aromatic heterocycles denote a ring system having 5 to 14 ring atoms (also denoted “heteroaryl” or “hetaryl”), preferably 5 to 10 ring atoms, where 1 or more ring atoms are an element other than carbon, e.g. N, O or S, as such or in combination. Preferred heteroaryls contain 5 or 6 ring atoms. The heteroaryls may be substituted, where appropriate, at one or more ring systems. Examples of suitable heteroaryls are mentioned above.

In the sense of the invention, all residues are considered combinable with one another unless stated otherwise in the definition of the residues. All conceivable subgroups thereof shall be considered disclosed.

The invention also relates to physiologically compatible salts of the compounds of general Formula (1). The physiologically compatible salts are obtained as usual by reaction of basic compounds of general Formula (1) with inorganic or organic salts or acids and by neutralization with inorganic or organic bases. Hydrochloric acid, sulphuric acid, nitric acid or hydrobromic acid are preferably used as inorganic acids and e.g. formic acid, acetic acid, propionic acid, glycolic acid, lactic acid, amygdalic acid, tartaric acid, malic acid, citric acid, malonic acid, maleic acid, fumaric acid, succinic acid, alginic acid, benzoic acid, 2-, 3- and 4-alkyloxy and acyloxy benzoic acids, ascorbic acid, C₁-C₃ alkylsulfonic acids, benzenesulfonic acid, nicotinic acid, isonicotinic acid and amino acids are used as organic acids. For example, ammonia, sodium hydroxide, and potassium hydroxide solution are used as inorganic bases and alkylamines, pyridine, quinoline, isoquinoline, piperazine and derivatives thereof, and picolines, quinaldine or pyrimidine are used as organic bases. In addition, physiologically compatible salts of the compounds according to general Formula (1) can be obtained by converting the substances which have as substituents a tertiary amino group in a basically known manner with alkylating agents—such as alkyl or aralkyl halides—into the corresponding quaternary ammonium salts.

The invention also relates to solvates of the compounds, including the pharmaceutically acceptable salts, acids, bases and esters as well as the active metabolites thereof and, where appropriate, the tautomers thereof according to general Formula (1) including prodrug formulations. Prodrug formulations here comprise all substances which are formed by simple transformation including hydrolysis, oxidation or reduction either enzymatically, metabolically or in any other way. A suitable prodrug contains e.g. a substance of general Formula (1) bound via an enzymatically cleavable linker (e.g. carbamate, phosphate, N-glycoside or a disulfide group) to a dissolution-improving substance (e.g. tetraethylene glycol, saccharides, formic acids or glucuronic acid, etc.). Such a prodrug of a compound according to the invention can be applied to a patient, and this prodrug can be transformed into a substance of general Formula (1) so as to obtain the desired pharmacological effect.

The term “pharmaceutically acceptable” indicates that the substance or composition is compatible chemically and/or toxicologically, with the other ingredients comprising a formulation, and/or the mammal being treated therewith.

The present invention further provides a pharmaceutical composition, which comprises a compound of Formula (1) or a pharmaceutically acceptable salt thereof, as defined hereinabove. In one embodiment, the pharmaceutical composition includes the compound of Formula (1) together with a pharmaceutically acceptable excipient, diluent or carrier.

The present invention further provides a compound of Formula (1) or a pharmaceutically acceptable salt thereof, for use in therapy.

The compounds according to the invention can be administered in different ways, e.g. topically, orally, parenterally, cutaneously, subcutaneously, intravenously, intramuscularly, rectally, or by inhalation. The oral or intravenous administration is preferred. The compound is given to a patient who needs a therapy for a disease coming under the indication spectrum of the compounds according to the invention over a period to be determined by a physician. The compound can be administered to both humans and other mammalian animals.

The dosage of the compounds according to the invention is determined by the physician on the basis of the patient-specific parameters, such as age, weight, sex, severity of the disease, etc. The dosage is preferably from 0.001 mg/kg to 1000 mg/kg body weight, preferably from 0.001 to 500 mg/kg body weight and most preferably from 0.001 to 100 mg/kg body weight.

Corresponding to the kind of administration, the medicament is suitably formulated, e.g. in the form of solutions or suspensions, simple tablets or dragees, hard or soft gelatine capsules, suppositories, ovules, preparations for injection, which are prepared according to common galenic methods.

The compounds according to the invention can be formulated, where appropriate, together with further active substances and with excipients common in pharmaceutical compositions, e.g.—depending on the preparation to be produced—talcum, gum arabic, lactose, starch, magnesium stearate, cocoa butter, aqueous and non-aqueous carriers, fatty bodies of animal or vegetable origin, paraffin derivatives, glycols (in particular polyethylene glycol), various plasticizers, dispersants or emulsifiers, pharmaceutically compatible gases (e.g. air, oxygen, carbon dioxide, etc.), preservatives.

In order to produce liquid preparations, additives and/or solvents, such as sodium chloride solution, DSMO, ethanol, acetone, sorbitol, glycerine, olive oil, almond oil, propylene glycol or ethylene glycol, can be used. For example, DMSO is an organic solvent and belongs to the class of sulfoxides. It is miscible with water and various organic solvents in any ratio. DMSO is characterized by a pharmaceutical effect (antiphlogistic and analgetic).

Since it was recognized that several compounds of the present invention have a low solubility and are (highly) hydrophobic, a solubilizing agent may be useful. A solubilizing agent is a non-ionic surface-active substance (surfactant). According to the present invention, preferred solubilizing agents are selected from PEG-hydrated castor oil (Cremophor®-series), D-tocopherol polyethylene glycol 1000-succinate (Kolliphor® TPGS), polysorbate 20 (Tween® 20), polysorbate 80 (Tween® 80), polyoxyethylated 12-hydroxystearic acid (Kolliphor® HS 15; Solutol® HS15), sorbitan monooleate (Span® 20), poloxamer 407 (Kolliphor® P 407), poloxamer 188 (Kolliphor® P 188), PEG 300 caprylic acid/capric glycerides (Softigen® 767), PEG 400 caprylic acid/capric glycerides (Labrasol), PEG 300 oleic acid glycerides (Labrafil® M-1944CS), PEG 300 linoleic acid glycerides (Labrafil® M-2125CS), lauroyl polyoxyglycerides (Gellusire® 44/14), n-decanoic acid ester with 1,2,3-propane trioloctanoate (Softigen® 767), mono- or di-fatty acid esters of PEG 330, 400 or 1750.

Commercial products of PEG hydrated castor oil are for example known on the basis of PEG7, PEG25, PEG35, PEG40, PEG50 and PEG60: Crodure™ 25 (Croda), Croduret™ 40 (Croda), Croduret™ 50 (Croda), Croduret™ 60 (Croda), Cremophor® RH 40 (BASF), Cremophor® RH 60 (BASF), Cremophor® RH 410 (BASF), Kolliphor EL or Cremophor EL (BASF), Emulgin® HRE 40 (BASF), Emulgin® HRE 455 (BASF) und EMAROL H40 (CISME, Italy). According to the present invention, PEG35 hydrated castor oil (Cremophor® EL) is particularly suitable.

When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The ready-made solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

Furthermore, inhalation compositions, e.g. in the form of aerosols, sprays or as micronized powder, are preferably produced. For this purpose, the compounds according to the invention are either dissolved or suspended in pharmaceutically conventional solvents and finely divided by means of excess pressure in a certain volume and inhaled. The procedure is made correspondingly in the solid substances to be inhaled which are also finely divided by means of excess pressure and inhaled. Other applicators working by means of excess pressure are also included here.

The invention also relates to pharmaceutical preparations which contain a therapeutically active amount of the active ingredients (compound according to the invention of Formula (1)) together with organic or inorganic solid or liquid, pharmaceutically compatible carriers which are suited for the intended administration and which interact with the active ingredients without drawbacks.

The compounds of the Formula (1) are believed to be novel and are provided as further aspects of the invention.

The ability of the compounds of the present invention to act as TrkA inhibitors has been demonstrated by the assays in the Examples of the present application.

Certain compounds which are inhibitors of TrkA are particularly useful in the treatment of multiple types of pain including inflammatory pain, neuropathic pain, and pain associated with cancer, surgery, and bone fracture.

In one embodiment, compounds of Formula (1) are useful for treating pain, including chronic and acute pain in a mammal.

Acute pain, as defined by the International Association for the Study of Pain, results from disease, inflammation, or injury to tissues. This type of pain generally comes on suddenly, for example, after trauma or surgery, and may be accompanied by anxiety or stress. The cause can usually be diagnosed and treated, and the pain is confined to a given period of time and severity. In some rare instances, it can become chronic.

Chronic pain, as defined by the International Association for the Study of Pain, is widely believed to represent disease itself. It can be made much worse by environmental and psychological factors. Chronic pain persists over a longer period than acute pain and is resistant to most medical treatments, generally over 3 months or more. It can and often does cause severe problems for patients.

Compounds of Formula (1) are also useful for treating or preventing cancer in a mammal. Examples of cancer types to be treated or prevented are pancreatic tumors, prostate tumors, lung tumors, kidney tumors, bladder tumors, liver tumors, lymphoma, leukemia (e.g. myeloid leukemia), oesophageal tumors, ovarian tumors, oral tumors, thyroid tumors, cervical tumors, head-and-neck tumors, breast tumors, neuroblastoma, gastric tumors, colon tumors, brain tumors (e.g. glioblastoma or medulloblastoma) and skin tumors (e.g. melanoma).

Compounds of Formula (1) are also useful for treating inflammation in a mammal.

Accordingly, the present invention is further related to a method of treating or preventing pain in a mammal, comprising administering to said mammal one or more compounds of Formula (1) or a pharmaceutically acceptable salt thereof in an amount effective to treat or prevent said pain. In one embodiment, the pain is chronic pain. In one embodiment, the pain is acute pain. In one embodiment, the pain is inflammatory pain. In one embodiment, the pain is neuropathic pain. In one embodiment, the pain is pain associated with cancer. In one embodiment, the pain is pain associated with surgery. In one embodiment, the pain is pain associated with bone fracture. In one embodiment, the method comprises a method of treating said pain in a mammal. In one embodiment, the method comprises a method of preventing said pain in a mammal.

The invention is further related to a method of treating or preventing inflammation in a mammal, comprising administering to said mammal one or more compounds of Formula (1) or a pharmaceutically acceptable salt thereof in an amount effective to treat or prevent said inflammation. In one embodiment, the method comprises a method of treating said inflammation in a mammal. In one embodiment, the method comprises a method of preventing said inflammation in a mammal.

The invention is further related to a method of treating or preventing cancer in a mammal, comprising administering to said mammal one or more compounds of Formula (1) or a pharmaceutically acceptable salt thereof in an amount effective to treat or prevent said cancer.

In one embodiment, the method comprises a method of treating said cancer in a mammal. In one embodiment, the method comprises a method of preventing said cancer in a mammal.

Compounds of Formula (1) may be administered alone as a sole therapy or can be administered in addition with one or more other substances and/or treatments that work by the same or a different mechanism of action. Examples include anti-inflammatory compounds, steroids (e.g., dexamethasone, cortisone and fluticasone), analgesics such as NSAIDs (e.g., aspirin, ibuprofen, indomethacin, and ketoprofen), and opioids (such as morphine), and chemotherapeutic agents. These agents may be administered with one or more compounds of Formula (1) as part of the same or separate dosage forms, via the same or different routes of administration, and on the same or different administration schedules according to standard pharmaceutical practice known to one skilled in the art.

In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonal antibodies.

Accordingly, the compounds of Formula (1) may be administered in combination with one or more agents selected from mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents, anti-androgens, targeted antibodies, HMG-CoA reductase inhibitors, and prenyl-protein transferase inhibitors. These agents may be administered with one or more compounds of Formula (1) as part of the same or separate dosage forms, via the same or different routes of administration, and on the same or different administration schedules according to standard pharmaceutical practice known to one skilled in the art.

As used herein, terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

In one embodiment, the terms “treatment” or “treating” as used herein, mean an alleviation, in whole or in part, of symptoms associated with a disorder or condition as described herein (e.g., multiple types of pain including inflammatory pain, neuropathic pain, and pain associated with cancer, surgery, and bone fracture), or slowing, or halting of further progression or worsening of those symptoms.

In one embodiment, the term “preventing” as used herein means the prevention of the onset, recurrence or spread, in whole or in part, of the disease or condition as described herein (e.g., multiple types of pain including inflammatory pain, neuropathic pain, and pain associated with cancer, surgery, and bone fracture), or a symptom thereof.

The terms “effective amount” and “therapeutically effective amount” refer to an amount of compound that, when administered to a mammal in need of such treatment, is sufficient to (i) treat or prevent a particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound of Formula (1) that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, horses, cows, rats, mice, hamsters, and primates, including humans.

Preferred compounds according to Formula (1) are:

| Compound | A | B |
|---|---|---|
| a1 | 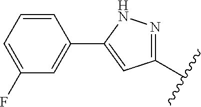 [SMILES: *c1cc(-c2cccc(F)c2)[nH]n1] | 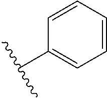 [SMILES: *c1ccccc1] |
| a3 | 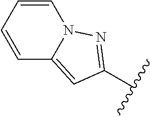 [SMILES: *c1cc2ccccn2n1] | 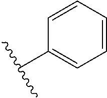 [SMILES: *c1ccccc1] |
| a4 | 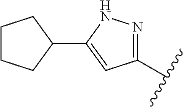 [SMILES: *c1cc(C2CCCC2)[nH]n1] | 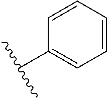 [SMILES: *c1ccccc1] |
| a8 | 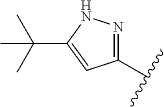 [SMILES: *c1cc(C(C)(C)C)[nH]n1] | 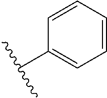 [SMILES: *c1ccccc1] |

(with C=as defined above for Formula (1); preferably: C=methylpiperazinyl)

| Compound | A | B |
|---|---|---|
| b1 | 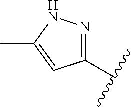 [SMILES: *c1cc(C)[nH]n1] | 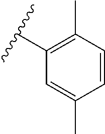 [SMILES: *c1cc(C)ccc1C] |
| b2 | 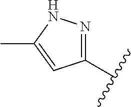 [SMILES: *c1cc(C)[nH]n1] | 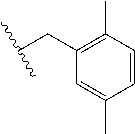 [SMILES: *Cc1cc(C)ccc1C] |
| b3 | 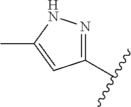 [SMILES: *c1cc(C)[nH]n1] | 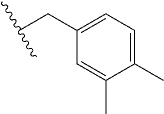 [SMILES: *Cc1ccc(C)c(C)c1] |
| b4 | 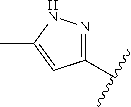 [SMILES: *c1cc(C)[nH]n1] | 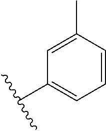 [SMILES: *c1cccc(C)c1] |
| b5 | 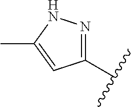 [SMILES: *c1cc(C)[nH]n1] | 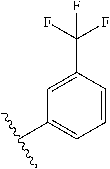 [SMILES: *c1cccc(C(F)(F)F)c1] |
| b6 | 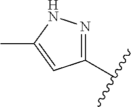 [SMILES: *c1cc(C)[nH]n1] | 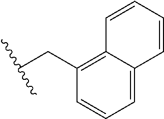 [SMILES: *Cc1cccc2ccccc12] |
| b7 | 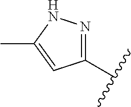 [SMILES: *c1cc(C)[nH]n1] | 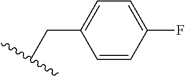 [SMILES: *Cc1ccc(F)cc1] |
| b8 | 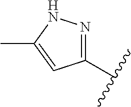 [SMILES: *c1cc(C)[nH]n1] | 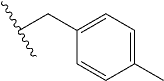 [SMILES: *Cc1ccc(C)cc1] |
| b9 | 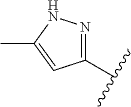 [SMILES: *c1cc(C)[nH]n1] | 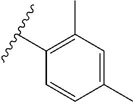 [SMILES: *c1ccc(C)cc1C] |

(with C=as defined above for Formula (1); preferably: C=methylpiperazinyl)

Particular preferred compounds according to the present invention are:

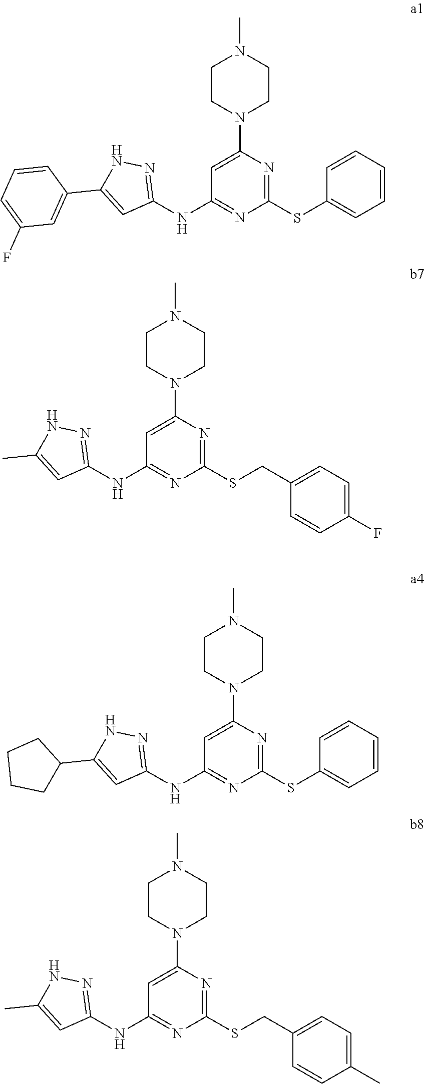

Particularly preferred is a1 (N-[5-(3-fluorophenyl)-1H-pyrazol-3-yl]-6-(4-methylpiperazin-1-yl)-2-(phenylsulfanyl)pyrimidin-4-amine). This means that in accordance with the above mentioned Formula (1) the substituent A is (3-fluorophenyl)-1H-pyrazol-3-yl, B is phenyl and C is methylpiperazinyl.

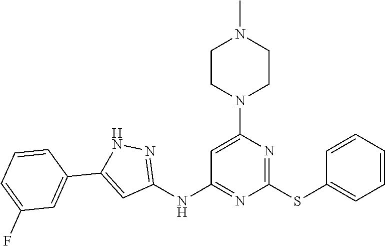 [SMILES: CN1CCN(c2cc(Nc3cc(-c4cccc(F)c4)[nH]n3)nc(Sc3ccccc3)n2)CC1]

For the sake of completeness it is mentioned that all general definitions given above in connection with Formula (1) compounds apply, of course, to each individual preferred compound, too. Thus, if a compound of Formula (1) is mentioned, each of the aforementioned preferred compounds is comprised, too.

Compounds in clinical trials provide a rich source for initiating new drug design efforts as for instance many compounds inhibit more than one molecular target and can have a therapeutic impact via so far unknown or ignored mechanisms. Following this idea, the inventors mined all kinases and corresponding inhibitors that had entered clinical trials and analyzed the respective protein binding sites with respect to selectivity determining features (a crucial element for the rational design of selective compounds). Based on these analyses, the target-compound pair tropomyosin receptor kinase A (TrkA) and tozasertib was selected as starting point for the design of improved TrkA inhibitors. Tozasertib (Originator Vertex Pharma) is in clinical trials for aurora kinase A (AurA), but also inhibiting TrkA, which is a validated drug target for cancer and pain. However, the selectivity of tozasertib for TrkA is not very high and its use is accompanied by several severe side effects, e.g. neutropenia, diarrhea, mucositis. In line with these side effects, the inhibition of the AurA has common adverse effects such as neutropenia and hematological toxicities (Falchook, G. S., Bastida, C. C. & Kurzrock, R. Aurora kinase inhibitors in oncology clinical trials: Current state of the progress. Semin. Oncol. 42, 832-848 (2015)). Thus, the inventors decided to switch the selectivity of tozasertib, originally developed against AurA as cancer target, towards the pain target TrkA.

The design of improved TrkA inhibitors itself was achieved by jointly employing a proprietary de novo design platform and a multi-objective selection scheme that considers selectivity and activity aspects as predicted by the also proprietary in silico tools. First, selectivity-determining features in the TrkA structure were identified (mentioned also above) by a novel approach called ‘selectivity grids’, where atom-based interaction energy grids of several TrkA, Aurora A and B structures were fused into one content-rich representation of target specific sub-pockets. This step highlighted three areas of interest for compound optimization: two favorable hydrophobic sub-pockets for TrkA-selectivity were identified adjacent to the gatekeeper residue and enclosed between the Asp residue of the DFG-motif and a Phe residue of the glycine-rich loop (G-loop), respectively, and one unfavorable pocket for TrkA-selectivity was identified that overlaps with the cyclopropyl moiety of tozasertib. The identified selectivity-determining areas subsequently guided the compound library design where the two function groups A and B in Formula 1 were enumerated using commercially available drug-like fragments and retrosynthetic rules. The resulting library was subsequently prioritized using a multi-objective compound selection scheme that filters for selective and highly active compounds. Key aspects of the filtering process included I) that the final selection of compounds was obtained in an automated fashion based on the scoring ranks (i.e., without any manual selection beside synthesizability criteria) and that II) the highly conserved and large target class of kinase was considered in addition to AurA as primary off-target.

In detail, the multi-objective compound selection scheme included the following steps. Initially, binding poses of the compounds were generated via docking calculations. Compounds were removed for reasons of either poor docking scores, wrong orientation, or lack of key interactions. In the next step, compounds with an unfavorable selectivity profile were filtered out. This was accomplished via machine learning-based activity prediction models (Merget, B., Turk, S., Eid, S., Rippmann, F. & Fulle, S. Profiling prediction of kinase inhibitors: Toward the virtual assay. J. Med. Chem. 60, 474-485 (2017)) that were used I) to remove promiscuous compounds (i.e., predicted to be active at IC50 of 500 nM on ≥20 kinases) and II) those that are predicted to be highly active (IC50<10 nM) on Aurora A, B, or C kinases. The selectivity filtering was complemented by a structure-based procedure employing the TrkA-Aurora ‘selectivity grids’ for rescoring of docking solutions. The remaining compounds were finally prioritized for highly active compounds using two complementary machine learning (ML) technologies. 1) All compounds were evaluated by an ‘MMP/ML’ approach that is trained on fragment-based Matched Molecular Pairs (MMPs) and quantifies compound activity differences (Turk, S., Merget, B., Rippmann, F. & Fulle, S. Coupling Matched Molecular Pairs with Machine Learning for virtual compound optimization, doi: 10.1021/acs.jcim.7b00298). II) In addition, compounds interacting with the Phe gatekeeper were additionally evaluated by a hybrid QM/ML pipeline. This pipeline is trained on high-level quantum mechanical (QM) calculations to quantify ligand-gatekeeper interactions and rescores the top hits in a second step with fragment molecular orbital calculations, taking the entire binding pocket into consideration. The final selection of compounds was obtained in an automated fashion based on the scoring ranks (i.e., without any manual selection beside synthesizability criteria). Overall, the employed pipeline resulted into a new compound series of TrkA inhibitors with an improved selectivity profile across the kinome compared to the starting compound tozasertib. The most preferred compound a1 is highly active on TrkA (pKd=8.6), has nanomolar cellular potency (26 nM) and high selectivity in the kinome panel (Selectivity Score=0.08 @100 nM and <35% ctrl as activity cut-off) (Tables 1-4). This was achieved by removing the cyclopropylcarboxamide tail of tozasertib and modifying the amino-5-methylpyrazole (best hits: a1 and a4).

The substitution at the 5- or 6-membered alkyl ring or alkenyl or aryl ring (e.g. via a fluorine as present in a1) seems to be an important step to achieve TrkA versus AurA selectivity. Analogues of tozasertib have not been employed for pain treatment so far. Modifying part B (i.e. N-(4-aminothiophene)cyclopropylcarboxamide) also resulted in two hits (b7 and b8) but with lower kinome selectivity compared to a1.

The particular preferred compound a1 was subjected to a pharmacokinetic evaluation in mice. The formulations contained compound a1 in DMSO together with a solubilizing agent of the Cremophor® series (e.g. Cremophor® EL) and NaCl. In this regard reference is made to Example 6 and FIGS. 2 and 3. In the experiments it has been clearly shown that, despite a certain expectation that compound a1 may be toxic, none of the animals showed any toxic sign. Thus, it was concluded that at least compound a1 may be a suitable candidate for forthcoming clinical trials.

The invention is further described with respect to the figures which show:

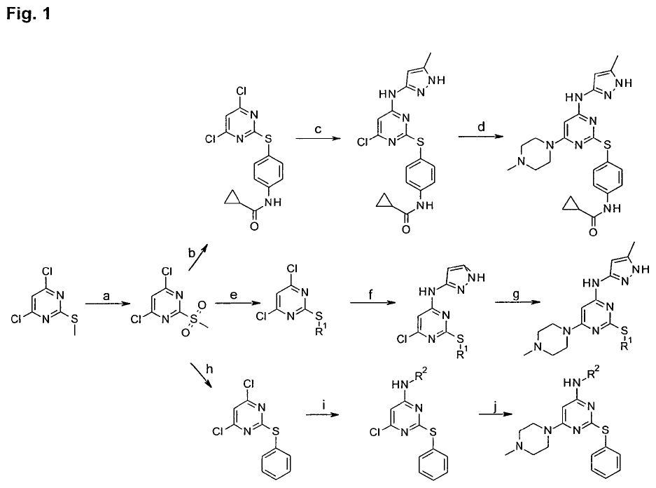

FIG. 1: General synthesis route for tozasertib analogues

The invention is further described in the following examples which are not construed to limit the invention.

### EXAMPLES

### Example 1: Method for the Preparation of Tozasertib and Tozasertib Analogues Having Improved TrkA Activity

The general synthetic route for the preparation of tozasertib (a-d) and tozasertib analogs (B: a, e-g; A: a, h-j) is shown in FIG. 1.

Reagents and conditions: (a) 3-chloroperoxybenzoic acid, DCM, rt, 3 h; (b) N-(4-mercaptophenyl)cyclopropanecarboxamide, TEA, CH₃CN, 80° C., 3-10 h; (c) 5-methyl-1H-pyrazol-3-amine, DIPEA, DMF, 95° C., 16 h; (d) amine (1-methylpiperazine or morpholine), DMF, DIPEA, 90° C., 6-12 h; (e) corresponding thiol, TEA, CH₃CN, 80° C., 3-10 h; (f) 5-methyl-1H-pyrazol-3-amine, DIPEA, dioxane, 95° C., 3-6 h, (g) amine (1-methylpiperazine or morpholine), DMF, DIPEA, 90° C., 6-12 h; (h) thiophenol, TEA, THF, 50° C.; (i) corresponding amine, DIPEA, dioxane, 95° C., 3-6 h; (j) amine (1-methylpiperazine or morpholine), DMF, DIPEA, 90° C., 6-12 h.

The structures and batch purities (290%) of the compounds have been confirmed by using standard analytical methods (LC/MS and NMR).

Analytical data (NMR and LC/MS)

### Tozasertib: N-[4-({4-[(5-methyl-1H-pyrazol-3-yl)amino]-6-(4-methylpiperazin-1-yl)pyrimidin-2-yl}sulfanyl)phenyl]cyclopropanecarboxamide

¹H NMR (400 MHz, DMSO-d₆) δ 0.8 (4H, CH₂), 1.8 (1H, C(═O)CH), 2.0 (3H, ArCH₃), 2.2 (3H, NCH₃), 2.3 (4H, N(CH₂)CH₂), 3.3 (4H, ArN(CH₂)CH₂), 5.4 (1H, ArNHAr), 6.0 (1H, ArH), 7.4 (2H, ArH), 7.7 (2H, ArH), 9.2 (1H, ArH), 10.4 (1H, ArNHCO), 11.7 (1H, ArH). LCMS purity 100%.

### Reference 2: N-(5-methyl-1H-pyrazol-3-yl)-6-(4-methylpiperazin-1-yl)-2-(phenylsulfanyl)-pyrimidin-4-amine

1H NMR (400 MHz, DMSO-d6) δ 2.0 (3H, ArCH3), 2.2 (3H, NCH3), 2.3 (4H, N(CH2)CH2), 3.3 (4H, ArN(CH2)CH2), 5.5 (1H, ArNHAr), 6.1 (1H, ArH), 7.5 (3H, ArH), 7.6 (2H, ArH), 9.2 (1H, ArH), 11.7 (1H, ArH). LCMS purity 100%.

### a1: N-[5-(3-fluorophenyl)-1H-pyrazol-3-yl]-6-(4-methylpiperazin-1-yl)-2-(phenylsulfanyl)pyrimidin-4-amine

1H NMR (400 MHz, DMSO-d6) δ 2.2 (3H, NCH3), 2.3 (4H, N(CH2)CH2), 3.4 (4H, ArN(CH2)CH2), 6.2 (2H, ArNHAr, ArH), 7.2 (1H, ArH), 7.4 (6H, ArH), 7.6 (2H, ArH), 9.5 (1H, ArH), 9.3 (1H, ArH), 12.7 (1H, ArH). LCMS purity 100%.

### a3: 6-(4-methylpiperazin-1-yl)-2-(phenylsulfanyl)-N-(pyrazolo[1,5-a]pyridin-2-yl)pyrimidin-4-amine

1H NMR (400 MHz, DMSO-d6) δ 2.2 (3H, NCH3), 2.3 (4H, N(CH2)CH2), 3.4 (4H, ArN(CH2)CH2), 5.8 (1H, ArH), 6.1 (1H, ArNHAr), 6.6 (1H, ArH), 7.1 (1H, ArH), 7.5 (2H, ArH), 7.6 (3H, ArH), 8.4 (1H, ArH), 9.8 (1H, ArH). LCMS purity 100%. LCMS purity 98.5%.

### a4: N-(5-cyclopentyl-1H-pyrazol-3-yl)-6-(4-methylpiperazin-1-yl)-2-(phenylsulfanyl)pyrimidin-4-amine

¹H NMR (400 MHz, DMSO-de) 61.4 (2H, cyclopentyl-H), 1.6 (4H, cyclopentyl-H), 1.9 (2H, cyclopentyl-H), 2.2 (3H, NCH₃), 2.3 (4H, N(CH₂)CH₂), 2.9 (1H, 2H, cyclopentyl-H), 5.6 (1H, ArNHAr), 6.3 (1H, ArH), 7.4 (3H, ArH), 7.6 (2H, ArH), 9.3 (1H, ArH), 11.9 (1H, ArH). LCMS purity 100%.

### a8: N-(5-tert-butyl-1H-pyrazol-3-yl)-6-(4-methylpiperazin-1-yl)-2-(phenylsulfanyl) pyrimidin-4-amine

¹H NMR (400 MHz, DMSO-de) δ 1.2 (9H, CH₃), 2.2 (3H, NCH₃), 2.3 (4H, N(CH₂)CH₂), 5.7 (1H, ArNHAr), 6.5 (1H, ArH), 7.5 (5H, ArH), 9.2 (1H, ArH), 11.9 (1H, ArH). LCMS purity 100%.

### b1: 2-[(2,5-dimethylphenyl)sulfanyl]-N-(5-methyl-1H-pyrazol-3-yl)-6-(4-methylpiperazin-1-yl)pyrimidin-4-amine

¹H NMR (400 MHz, DMSO-de) δ 2.0 (3H, ArCH₃), 2.2 (6H, NCH₃, ArCH₃), 2.4 (3H, ArCH₃), 2.6 (4H, N(CH₂)CH₂), 3.4 (4H, ArN(CH₂)CH₂), 5.4 (1H, ArNHAr), 6.0 (1H, ArH), 7.2 (2H, ArH), 7.4 (1H, ArH), 9.2 (1H, ArH), 11.7 (1H, ArH). LCMS purity 95.8%.

### b2: 2-{[(2,5-dimethylphenyl)methyl]sulfanyl}-N-(5-methyl-1H-pyrazol-3-yl)-6-(4-methylpiperazin-1-yl)pyrimidin-4-amine

¹H NMR (400 MHz, DMSO-de) δ 2.1 (3H, ArCH₃), 2.2 (6H, 2×ArCH₃), 2.3 (3H, NCH₃), 2.4 (4H, N(CH₂)CH₂), 3.5 (4H, ArN(CH₂)CH₂), 4.3 (2H, CH₂), 5.9 (1H, ArNHAr), 6.4 (1H, ArH), 7.0 (1H, ArH), 7.1 (1H, ArH), 7.2 (1H, ArH), 9.2 (1H, ArH), 11.9 (1H, ArH). LCMS purity 91.8%.

### b3: 2-{[(3,4-dimethylphenyl)methyl]sulfanyl}-N-(5-methyl-1H-pyrazol-3-yl)-6-(4-methylpiperazin-1-yl)pyrimidin-4-amine

¹H NMR (400 MHz, DMSO-de) 2.2 (12H, 3×ArCH₃, NCH₃), 2.4 (4H, N(CH₂)CH₂), 3.5 (4H, ArN(CH₂)CH₂), 4.3 (2H, CH₂), 5.9 (1H, ArNHAr), 6.5 (1H, ArH), 7.0 (1H, ArH), 7.1 (1H, ArH), 7.2 (1H, ArH), 9.2 (1H, ArH), 11.9 (1H, ArH). LCMS purity 98.1%.

### b4: N-(5-methyl-1H-pyrazol-3-yl)-2-[(3-methylphenyl)sulfanyl]-6-(4-methylpiperazin-1-yl)pyrimidin-4-amine

¹H NMR (400 MHz, DMSO-de) δ 2.0 (3H, ArCH₃), 2.2 (3H, NCH₃), 2.3 (7H, ArCH₃, N(CH₂)CH₂), 3.3 (4H, ArN(CH₂)CH₂), 5.5 (1H, ArNHAr), 6.1 (1H, ArH), 7.3 (5H, ArH), 9.2 (1H, ArH), 11.7 (1H, ArH). LCMS purity 91.1%.

### b5: N-(5-methyl-1H-pyrazol-3-yl)-6-(4-methylpiperazin-1-yl)-2-{[3-(trifluoromethyl) phenyl]sulfanyl}pyrimidin-4-amine

¹H NMR (400 MHz, DMSO-de) δ 2.0 (3H, ArCH₃), 2.1 (3H, NCH₃), 2.3 (4H, N(CH₂)CH₂), 3.3 (4H, ArN(CH₂)CH₂), 5.5 (1H, ArNHAr), 6.2 (1H, ArH), 7.7 (1H, ArH), 7.8 (2H, ArH), 7.9 (1H, ArH), 9.3 (1H, ArH), 11.8 (1H, ArH). LCMS purity 95.8%.

### b6: N-(5-methyl-1H-pyrazol-3-yl)-6-(4-methylpiperazin-1-yl)-2-[(naphthalen-1-ylmethyl)sulfanyl]pyrimidin-4-amine

¹H NMR (400 MHz, DMSO-de) δ 2.1 (3H, ArCH₃), 2.2 (3H, NCH₃), 2.3 (4H, N(CH₂)CH₂), 3.5 (4H, ArN(CH₂)CH₂), 4.8 (2H, CH₂), 5.9 (1H, ArNHAr), 6.5 (1H, ArH), 7.3 (1H, ArH), 7.6 (2H, ArH), 7.7 (1H, ArH), 7.8 (1H, ArH), 8.0 (1H, ArH), 8.2 (1H, ArH), 9.3 (1H, ArH), 11.9 (1H, ArH). LCMS purity 100%.

### b7: 2-{[(4-fluorophenyl)methyl]sulfanyl}-N-(5-methyl-1H-pyrazol-3-yl)-6-(4-methylpiperazin-1-yl)pyrimidin-4-amine

¹H NMR (400 MHz, DMSO-de) δ 2.2 (6H, ArCH₃, NCH₃), 2.3 (4H, N(CH₂)CH₂), 3.5 (4H, ArN(CH₂)CH₂), 4.3 (2H, CH₂), 5.9 (1H, ArNHAr), 6.5 (1H, ArH), 7.1 (2H, ArH), 7.4 (2H, ArH), 9.2 (1H, ArH), 11.9 (1H, ArH). LCMS purity 95.6%.

### b8: N-(5-methyl-1H-pyrazol-3-yl)-2-{[(4-methylphenyl)methyl]sulfanyl}-6-(4-methylpiperazin-1-yl)pyrimidin-4-amine

¹H NMR (400 MHz, DMSO-de) δ 2.1 (9H, 2×ArCH₃, NCH₃), 2.3 (4H, N(CH₂)CH₂), 3.4 (4H, ArN(CH₂)CH₂), 4.3 (2H, CH₂), 5.8 (1H, ArNHAr), 6.4 (1H, ArH), 7.1 (2H, ArH), 7.2 (2H, ArH), 9.2 (1H, ArH), 11.9 (1H, ArH). LCMS purity 92.8%.

### b9: 2-[(2,4-dimethylphenyl)sulfanyl]-N-(5-methyl-1H-pyrazol-3-yl)-6-(4-methylpiperazin-1-yl)pyrimidin-4-amine

¹H NMR (400 MHz, DMSO-de) δ 2.0 (3H, ArCH₃), 2.2 (3H, NCH₃), 2.3 (10H, N(CH₂)CH₂ & ArCH₃), 3.3 (4H, ArN(CH₂)CH₂), 5.4 (1H, ArNHAr), 6.0 (1H, ArH), 7.1 (1H, ArH), 7.2 (1H, ArH), 7.4 (1H, ArH), 9.2 (1H, ArH), 11.7 (1H, ArH). LCMS purity 95.4%.

### Example 2: Primary Screen

Compounds were initially tested in a primary screen against TrkA (at 10 and 100 nM) and AurA (at 100 nM and 1 μM) using the KINOMEscan technology from DiscoverX Corporation, Fremont, Calif., USA (https://www.discoverx.com/services/drug-discovery-development-services/kinase-profiling/kinomescan).

Results were reported as % of control (% Ctrl.=(test compound signal−positive control signal)/(DMSO signal−positive control signal). KINOMEscan is an active site-directed competition binding assay that quantitatively measures the ability of a compound to compete with an immobilized, active-site directed ligand.

TABLE 1
Results of primary screens of biological assay.

| | |
|---|---|
| % Ctrl. TrkA | % Ctrl. AurA |

| ID | @10 nM | @100 nM | @100 nM | @1 μM |
|---|---|---|---|---|
| Tozasertib | 31 | 5.2 | 0.4 | 0 |
| Reference 2 | 58 | 10 | 15 | 0.95 |
| a1 | 11 | 1.9 | 97 | 92 |
| a3 | 93 | 29 | 87 | 76 |
| a4 | 15 | 0.95 | 49 | 4.5 |
| a8 | 69 | 18 | 100 | 79 |
| b1 | 80 | 11 | 7.3 | 0.5 |
| b2 | 93 | 31 | 54 | 9.4 |
| b3 | 53 | 11 | 46 | 4.5 |
| b4 | 50 | 6.7 | 2.6 | 0.1 |
| b5 | 90 | 32 | 36 | 2.4 |
| b6 | 74 | 35 | 63 | 7.4 |
| b7 | 9.3 | 0.5 | 15 | 0.85 |
| b8 | 35 | 5.3 | 30 | 1.3 |
| b9 | 77 | 24 | 51 | 3.8 |

¹ Compound “Reference 2”: Tozasertib without cyclopropylcarboxamide tail

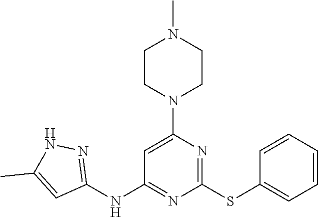 [SMILES: Cc1cc(Nc2cc(N3CCN(C)CC3)nc(Sc3ccccc3)n2)n[nH]1]

Reference 2 has compared to tozasertib an improved selectivity towards AurA (i.e. ΔΔpKd=1; Table 2) pointing to the contribution of removing the cyclopropylcarboxamide tail in addition to the modifications in compound part A.

### Example 3: Kd Measurement

Kd measurements were done via the KdELECT product service from DiscoverX Corporation (Freemont, Calif., USA) which also employs the KINOMEscan technology. Here, inhibitor binding constants (Kd values) are calculated from duplicate 11-point dose-response curves.

TABLE 2
Experimental Kd/pKd values of top hits.

| | TrkA | AurA | TrkA | AurA | |
|---|---|---|---|---|---|
| ID | [nM] | [nM] | [pKd] | [pKd] | ΔpKd |
| Tozasertib | 3.4 | 0.47 | 8.5 | 9.3 | −0.8 |
| Reference | 30 | 41 | 7.5 | 7.4 | 0.1 |
| 2 | | | | | |
| a1 | 2.6 | 4500 | 8.6 | 5.3 | 3.3 |
| a4 | 1.5 | 93 | 8.8 | 7.0 | 1.8 |
| b7 | 1.1 | 28 | 9.0 | 7.6 | 1.4 |
| b8 | 3.8 | 53 | 8.4 | 7.3 | 1.1 |

### Example 4: Selectivity Profiling

Selectivity profiling was done over a panel of 97 kinases at 100 nM concentration (scanEDGE assay panel from Discover X, which also uses the KINOMEscan technology, whereat KIT(D816V) and KIT (V559D, T6701) were replaced by TrkB and TrkC). The best compound (a1) was additionally profiled at 1 μM concentration using the same panel. The employed profiling panel consisted of the following kinases: ABL1(T3151)-phosphorylated, ABL1-nonphosphorylated, ABL1-phosphorylated, ACVR1B, ADCK3, AKT1, AKT2, ALK, AURKA, AURKB, AXL, BMPR2, BRAF, BRAF (V600E), BTK, CDK11, CDK2, CDK3, CDK7, CDK9, CHEK1, CSF1R, CSNK1D, CSNK1G2, DCAMKL1, DYRK1B, EGFR, EGFR (L858R), EPHA2, ERBB2, ERBB4, ERK1, FAK, FGFR2, FGFR3, FLT3, GSK3B, IGF1R, IKK-alpha, IKK-beta, INSR, JAK2 (JH1domain-catalytic), JAK3 (JH1domain-catalytic), JNK1, JNK2, JNK3, KIT, LKB1, MAP3K4, MAPKAPK2, MARK3, MEK1, MEK2, MET, MKNK1, MKNK2, MLK1, p38-alpha, p38-beta, PAK1, PAK2, PAK4, PCTK1, PDGFRA, PDGFRB, PDPK1, PIK3C2B, PIK3CA, PIK3CG, PIM1, PIM2, PIM3, PKAC-alpha, PLK1, PLK3, PLK4, PRKCE, RAF1, RET, RIOK2, ROCK2, RSK2 (Kin. Dom. 1-N-terminal), SNARK, SRC, SRPK3, TGFBR1, TIE2, TRKA, TRKB, TRKC, TSSK1B, TYK2 (JH1domain-catalytic), ULK2, VEGFR2, YANK3, ZAP70

TABLE 3
Experimental profiling data @ 100 nM screening concentration.

| | # | Selectivity | |
|---|---|---|---|
| ID | Hits | Score | List of inhibited kinases |
| Tozasertib | 10 | 0.109 | TrkA, ABL1, AurA, AurB, AXL, |
| | | | CK1d, FLT3, JAK2, PLK4, RET |
| a1 | 7 | 0.076 | TrkA, TrkB, TrkC, FLT3, KIT, |
| | | | PDGFRb, RET |
| a4 | 19 | 0.207 | TrkA, TrkB, TrkC, ABL1, AurA, |
| | | | AXL, FGFR2, FGFR3, FLT3, FMS, |
| | | | INSR, JAK2, JAK3, KIT, MAP2K2, |
| | | | MET, PDGFRb, RET, SRC |
| b7 | 18 | 0.196 | TrkA, TrkB, TrkC, ABL1, ALK, |
| | | | AurA, FLT3, FMS, JAK2, JAK3, |
| | | | KDR, KIT, NuaK2, PDGFRb, PLK4, |
| | | | RET, SRC, TYK2 |
| b8 | 17 | 0.185 | TrkA, TrkB, TrkC, ABL1, AurA, |
| | | | AXL, BTK, FGFR3, FLT3, JAK2, |
| | | | JAK3, KDR, KIT, PLK4, RET, SRC, |
| | | | TYK2 |

Kinase panel: N = 92 non-mutant kinases; activity cut-off: <35% ctrl @ 100 nM
Selectivity Score = Number of inhibited kinases/Number of tested kinases
Experimental profiling data @ 1 μM screening concentration for a1: Selectivity Score = 0.207

### Example 5: Cellular Assay

The two compounds a1 and b7 were additionally tested in a functional assay using the PathHunter® technology (DiscoverX Corporation, Freemont, Calif., USA; https://www.discoverx.com/technologies-platforms/enzyme-fragment-complementation-technoloqy/cell-based-efc-assays/protein-protein-interactions/kinases-cell-based-(rtk-ctk)). In this assay, the full-length human TrkA receptor (i.e. including ligand-binding and kinase domains) is expressed as a C-terminal (intracellular) fusion with a small peptide epitope, called Prolink™ (PK). Furthermore, a SH2 domain containing protein (i.e. Shc1) is co-expressed with a larger Enzyme Acceptor (EA) fragment. Activation of the TrkA receptor by adding the agonist ligand (β-NGF), leads to the auto-phosphorylation of the kinase and subsequent binding of the Shc1-EA protein. That recruitment results in an active B-galactosidase enzyme that is detected by addition of a chemiluminescent substrate.

Compounds were tested for antagonism using EC80 concentration (i.e. 0.03 μg/ml) of β-NGF. Data was normalized to the maximal and minimal response observed in the presence of EC80 agonist ligand (3-NGF) and vehicle (DMSO, final concentration 1%), respectively.

TABLE 4
Compound activity in cell-based assay.

| ID | Cellular assay [RC50] |
|---|---|
| a1 | 26 nM |
| b7 | 23 nM |

### Example 6: Pharmacokinetic Evaluation of Compound a1 after Intravenous Administration in Mice

Compound a1 is highly active on TrkA, most importantly from a design perspective, has a 10,000-fold improved selectivity against the selected off-target AurA. Nanomolar cellular potency against TrkA underlines the potential value of compound a1 as an advanced hit compound for the treatment of acute and chronic pain or other conditions associated with abnormal TrkA activity such as inflammation and cancer.

Compound a1 was synthesized under contract by the company Enamine Ltd. according to Example 1. Dimethylsulfoxide (DMSO) from J. T. Baker (Germany), Cremophor EL (Polyoxyethyleneglycerol triricinoleate 35 castor oil) from BASF (Germany), and Saline solution 0.9% from B. Braun (Germany) were used in this study. Two types of formulations were prepared: a formulation with a high dose of compound a1 (9 mM) and a formulation with a low dose of compound a1 (1.8 mM); c.f. Tables 5 and 6. First, a 100 mM stock solution of compound a1 in DMSO was prepared by dissolving 18.46 mg of compound a1 (molecular weight of 461.56 g/mol) in 400 μl DMSO at room temperature. The formulation with the high dose of a1 was prepared using 9% a1-DMSO stock solution, 10% Cremophor EL, and 81% saline. The formulation with the low dose of a1 contained 1.8% a1-DMSO stock solution, 5% Cremophor EL, and 93.2% saline; c.f. FIG. 2. Both formulations were checked in serial dilutions in order to make sure that compound a1 did not precipitate after intravenous injection.

TABLE 5
Preparation of test solutions for i.v. injection in mice

| | | | vehi- | vehi- | |
|---|---|---|---|---|---|
| | a1_low | a1_high | cle_low | cle_high | saline |
| a1-DMSO stock | 1.8% | 9.0% | — | — | — |
| solution (100 mM) | | | | | |
| DMSO | — | — | 1.8% | 9.0% | — |
| Cremophor EL | 5.0% | 10.0% | 5.0% | 10.0% | — |
| NaCl 0.9% | 93.2% | 81.0% | 93.2% | 81.0% | 100% |

Experiments were carried out in accordance with the EU Directive 86/609/EEC on the protection of animals used for scientific purposes. Female adult C57Bl/6 mice (6-8 weeks old, body weight 20 g) were treated with 5 mg/kg (low dose) and 25 mg/kg (high dose) compound a1 or with vehicle (formulations without compound a1) or with saline by intravenous (i.v.) injections for 1 hour using a 1 ml syringe (29-G, Terumo); c.f. Table 6. After injection, the mice were returned to their home cages, and after 1 hour, the mice were anaesthetized with isoflurane. Terminal blood samples were collected using a 1 ml syringe (20-G needle, B. Braun). The blood was immediately transferred into a 1.3 ml heparin-coated tube (Sarstedt). Plasma was collected by centrifugation at 1500 g for 15 minutes. The supernatant was placed in a 1.5 ml tube on dry ice and stored at −20° C.

The quantification of compound a1 in murine plasma samples was performed via liquid chromatography tandem mass spectrometry (LC-MS-MS).

TABLE 6
Pharmacokinetic evaluation of compound
a1 after i.v. injection in mice

| | | | | Plasma Conc. |
|---|---|---|---|---|
| Animal No. | | Injection | | of Compound |
| (C57BI/6 mice) | Test Item | Volume | Toxicity | a1 |
| 1 | a1_low | 150 μl | None | 407 nM |
| | (5 mg/kg) | | | |
| 2 | a1_low | 150 μl | None | 272 nM |
| | (5 mg/kg) | | | |
| 3 | a1_high | 150 μl | None | 805 nM |
| | (25 mg/kg) | | | |
| 4 | a1_high | 150 μl | None | 920 nM |
| | (25 mg/kg) | | | |
| 5 | vehicle_low | 150 μl | None | n.d. |
| 6 | vehicle_low | 150 μl | None | n.d. |
| 7 | vehicle_high | 150 μl | None | n.d. |
| 8 | vehicle_high | 150 μl | None | n.d. |
| 9 | saline | 150 μl | None | n.d. |
| 10 | saline | 150 μl | None | n.d. |

n.d. = not detectable (below detection limit)

Results: The LC-MS-MS analysis confirmed an average concentration of compound a1 in plasma of mice which were treated for 1 hour with 5 mg/kg (low dose) and 25 mg/kg (high dose) of 340 nM and 863 nM, respectively. Surprisingly, none of the animals showed any obvious signs of toxicity (Table 6).

1. Compounds of Formula (1) 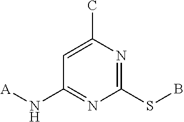
wherein
A: 5-membered monocyclic aromatic heterocyclic ring or 8-10 membered bicyclic aromatic heterocyclic ring each containing 2-4 nitrogen atoms and optionally one sulfur or oxygen atom, said ring optionally substituted (a) with one or more C₁-C₄ alkyl or alkenyl groups, or (b) with a 5- or 6-membered alkyl ring or alkenyl ring or aryl ring that are optionally substituted with one or more halogen or C₁-C₄ alkyl or alkenyl groups including mono- or poly-halogenated C₁-C₄ alkyl or alkenyl groups
B: methylene-aryl or aryl rings that are optionally substituted with one or more halogen or C₁-C₄ alkyl or alkenyl groups including mono- or poly-halogenated C₁-C₄ alkyl or alkenyl groups
C: Monocyclic or bicyclic, saturated or monounsaturated or polyunsaturated or aromatic heterocycles having 5-10 ring atoms among them 1-5 heteroatoms which are preferably N, O and S, substituted, where appropriate, once, twice or thrice with residues R^{§}
R^{§}: —OH, —SH, —C₁-C₄ alkyl, —O—C₁₋₈ alkyl, —O—C₆₋₁₄ aryl, —S—C₁₋₄ alkyl, —S—C₆₋₁₄ aryl, —SO—C₁₋₄ alkyl, —SO—C₆₋₁₄ aryl, —SO₂—C₁₋₄ alkyl, —SO₂—C₆₋₁₄ aryl, —SO₃H, —OSO₂C₁₋₈ alkyl, —OSO₂C₆₋₁₄ aryl, —COOH, —COOC₁₋₈ alkyl, —(CO)C₁₋₈ alkyl, —COOH, —CONH₂, —CONHC₁₋₆ alkyl, —CON(C₁₋₆ alkyl)₂, —NH₂, —NHC₁₋₆ alkyl, —N(C₁₋₆ alkyl)₂, —NHC₆₋₁₄ aryl, —NH-hetaryl, —N(C₆₋₁₄ aryl)₂, —N(C₁₋₆ alkyl)(C₆₋₁₄ aryl), —C₁₋₆ alkyl, —C₂₋₁₂ alkenyl, halogen, —CH₂CH₂OH, —CH₂CH₂SH, —CH₂CH₂SCH₃, -sulfamoyl, alkylsulfamoyl, dialkyl sulfamoyl, -sulfo, phosphono, —CN, —NO₂ and/or —SCN
as well as pharmaceutically compatible salts and solvates of these compounds.

2. A compound of claim 1, wherein C is a residue as defined in claim 1, and A and B are as follows:

| A | B |
|---|---|
| 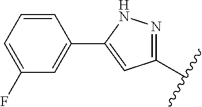 [SMILES: *c1cc(-c2cccc(F)c2)[nH]n1] | 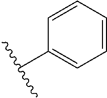 [SMILES: *c1ccccc1] |
| 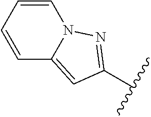 [SMILES: *c1cc2ccccn2n1] | 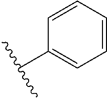 [SMILES: *c1ccccc1] |
| 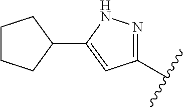 [SMILES: *c1cc(C2CCCC2)[nH]n1] | 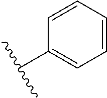 [SMILES: *c1ccccc1] |
| 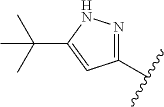 [SMILES: *c1cc(C(C)(C)C)[nH]n1] | 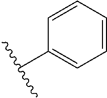 [SMILES: *c1ccccc1] |
| 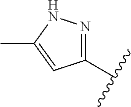 [SMILES: *c1cc(C)[nH]n1] | 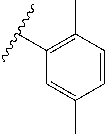 [SMILES: *c1cc(C)ccc1C] |
| 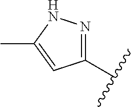 [SMILES: *c1cc(C)[nH]n1] | 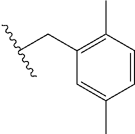 [SMILES: *Cc1cc(C)ccc1C] |
| 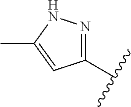 [SMILES: *c1cc(C)[nH]n1] | 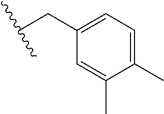 [SMILES: *Cc1ccc(C)c(C)c1] |
| 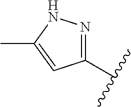 [SMILES: *c1cc(C)[nH]n1] | 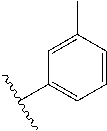 [SMILES: *c1cccc(C)c1] |
| 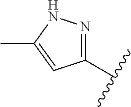 [SMILES: *c1cc(C)[nH]n1] | 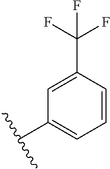 [SMILES: *c1cccc(C(F)(F)F)c1] |
| 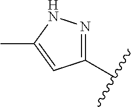 [SMILES: *c1cc(C)[nH]n1] | 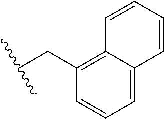 [SMILES: *Cc1cccc2ccccc12] |
| 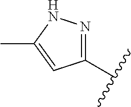 [SMILES: *c1cc(C)[nH]n1] | 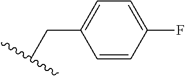 [SMILES: *Cc1ccc(F)cc1] |
| 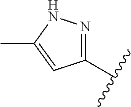 [SMILES: *c1cc(C)[nH]n1] | 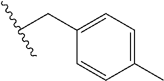 [SMILES: *Cc1ccc(C)cc1] |
| 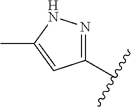 [SMILES: *c1cc(C)[nH]n1] | 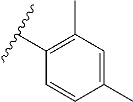 [SMILES: *c1ccc(C)cc1C] |

3. The compounds of claim 2, wherein C is methylpiperazinyl.

4. The compound of claim 1 having the following structures

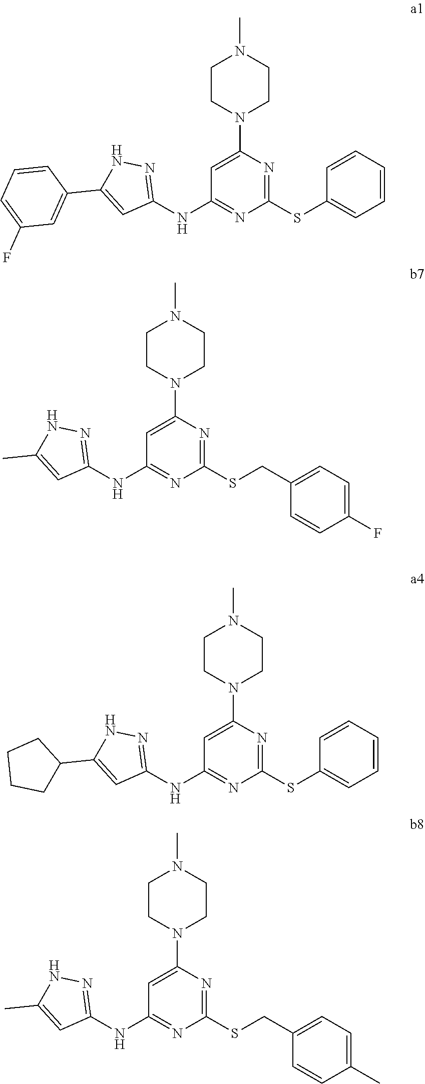

5. The compound of claim 4 being N-[5-(3-fluorophenyl)-1H-pyrazol-3-yl]-6-(4-methylpiperazin-1-yl)-2-(phenylsulfanyl)pyrimidin-4-amine)

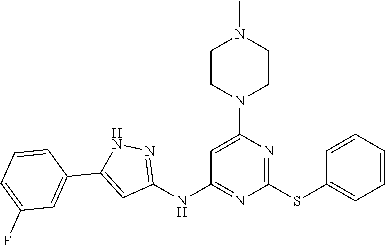 [SMILES: CN1CCN(c2cc(Nc3cc(-c4cccc(F)c4)[nH]n3)nc(Sc3ccccc3)n2)CC1]

6. A pharmaceutical composition comprising (a) the compound of claim 1 and/or a pharmaceutically acceptable salt thereof and (b) a pharmaceutically acceptable excipient.

7. The pharmaceutical composition of claim 6 in a form for topical, oral, parenteral, cutaneous, subcutaneous, intravenous and/or intramuscular administration.

8. The compound of claim 1 and/or a pharmaceutically acceptable salt thereof for use in a method of treating pain in a mammal.

9. The compound for the use of claim 8, wherein the pain is inflammatory pain, neuropathic pain, and pain associated with cancer, surgery, and bone fracture.

10. The compound for the use of claim 8, wherein the pain includes chronic and/or acute pain.

11. The compound of claim 1 and/or a pharmaceutically acceptable salt thereof for use in a method of treating cancer in a mammal.

12. The compound for the use of claim 11, wherein the cancer is chosen from pancreatic tumors, prostate tumors, lung tumors, kidney tumors, bladder tumors, liver tumors, lymphoma, leukemia, oesophageal tumors, ovarian tumors, oral tumors, thyroid tumors, cervical tumors, head-and-neck tumors, breast tumors, neuroblastoma, gastric tumors, colon tumors, brain tumors and skin tumors.
